(19) United States
(12) Patent Application Publication
(10) Pub. No.: US 2005/0203386 A1
(43) Pub. Date: Sep. 15, 2005

(54) Method of calibrating an X-ray imaging device

(75) Inventors: Benno Heigl, Coburg (DE); Alois Nottling, Pottenstein (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/076,532

(22) Filed: Mar. 8, 2005

(30) Foreign Application Priority Data
Mar. 11, 2004 (DE) 10 2004 012 057.9

(51) Int. Cl. G01N 23/04; A61B 5/05

(52) U.S. Cl. 600/427

(57) ABSTRACT

The present invention relates to an x-ray imaging device as well as a method for its calibration. With the method for calibrating the x-ray imaging device with at least two separate recording systems arranged for different recording planes (1 to 4) a 2D x-ray image (9, 10) of an area under examination is recorded during an intervention with an instrument (8) at any different positions of the instrument (8) with the two recording systems (1 to 4) simultaneously or immediately adjacent in time in order to obtain in number of pairs of images. From the 2D x-ray images (9, 10) a marker point (5) of the instrument (8) recognizable in the 2D x-ray images (9, 10) is extracted with an image processing algorithm. The image coordinates of the extracted point (5) of each pair of images are assigned to one another and from the assignments a relative imaging geometry of the two recording systems (1 to 4) is calculated. The present method makes do without any additional aids and can also be performed during the intervention.

### CROSS REFERENCE TO RELATED APPLICATIONS

This application claims priority to the German application No. 10 2004 012 057.9, filed Mar. 11, 2004 which is incorporated by reference herein in its entirety.

### FIELD OF INVENTION

The present invention relates to an x-ray imaging device as well as to a method for calibration of the x-ray imaging device which features at least two separate recording systems, arranged for different recording angles for recording 2D x-ray images of an area under examination, as well as to an evaluation and control unit for control of the recording systems and evaluation of the image data obtained by the recording systems.

### BACKGROUND OF INVENTION

X-ray imaging devices with two separate recording systems arranged for different recording planes are used above all as so-called biplane devices. These types of biplane devices feature C-arms arranged at an angle to each other, to each of which at least one x-ray source and one x-ray detector are attached opposite one another. By moving the recording system of a C-arm consisting of x-ray source and x-ray detector on the path predetermined by the C-arm, different projection directions of the area under examination can be recorded as 2D x-ray images. The two separate C-arms with their associated recording systems enable simultaneous recording in two different planes in this case.

Biplane devices are suitable for tasks such as imaging in surgical or interventional procedures, to locate the position of a medical instrument within the area under examination during the Intervention on the simultaneously recorded 2D x-ray images. Thus, especially with intravascular examinations with the aid of catheters, the x-ray images recorded simultaneously and displayed on a monitor are needed for the navigation of the catheter.

In many cases the precise geometrical imaging characteristics of the recording systems are required to enable the 2D x-ray images recorded with the two recording systems to be compared. These are needed for example for the calculation of biplane angiograms in the area of angiographic recording techniques. The imaging characteristics of the two recording systems in relation to one another can be determined by a calibration.

As a rule a biplane device has previously been calibrated by placing a calibration object with a known geometry within the recording area such that it is completely visible in the x-ray images of the two recording systems of the C-arms. In the 2D x-ray images the features of the calibration object are then extracted and uniquely assigned to the 3D structures of the calibration object. Through this arrangement the projection geometry of each recording system can be uniquely calculated in relation to the calibration object. These calibration steps are as a rule undertaken before the actual intervention is performed.

Such a method is known for example from DE 101 14 099 A1. This document primarily deals with a method in which the three-dimensional position of an instrument introduced into the body area is captured from two 2D x-ray images which were recorded at different angles with two separate recording systems, are inserted into a 3D representation from a previously recorded 3D data record.

U.S. Pat. No. 5,859,922 discloses a method for determining the three-dimensional position of a heart pacemaker wire in the heart of a patient. Here too, to determine the 3D position, two x-ray images are recorded from different projection directions with a biplane x-ray system. These requires a prior calibration of the x-ray system which, like the method disclosed in this document, is also undertaken with a specific calibration object.

Attaching markers to a patient for a calibration of the biplane device during the intervention is also known, designed to fulfill the same function as the features of the calibration object. The use of markers along the catheter is also known, in which case the catheter must then be directed to specific predetermined positions within the area under examination to obtain the assignment necessary for the calibration.

U.S. Pat. No. 6,047,080 relates to a method as well as an imaging system for three-dimensional reconstruction of the coronary tree from angiographic x-ray images. The publication describes in one alternative a method for calibrating the two recording systems of the x-ray imaging device used without the use of a calibration phantom. However this calibration requires a great deal of effort in image processing in which from the individual images initially by detection, segmentation and identification of central axes of the blood vessels a hierarchical representation of the image tree is created and subsequently branch points must be determined. From an assignment of the branch points in the individual images the relative imaging geometry can then be calculated. However at least five corresponding marker points are required in each image for this. Furthermore the method requires a great deal of interaction on the part of the user to avoid miscalibration.

### SUMMARY OF INVENTION

An object of the present invention is to specify a method for calibrating an x-ray imaging device as well as an x-ray imaging device embodied for this calibration with which the calibration can be executed in a simple way and without additional aids for the person operating the x-ray imaging.

The object is achieved by the claims. Advantageous embodiments of the method and of the x-ray imaging device are the object of the dependent claims or can be taken from the subsequent description as well as the exemplary embodiment.

With the present method for calibrating an x-ray imaging device, especially a biplane device with at least two separate recording systems arranged for different recording angles, during an intervention with for example an endoluminal instrument, especially a catheter, at any number of different positions of the instrument a 2D image is recorded with the two recording systems at the same time or immediately adjacent in time. Each 2D x-ray image which was recorded with one of the two recording systems in this case forms a pair along with the 2D x-ray image recorded at the same time or immediately adjacent in time with the other recording system. From the two 2D x-ray images of each image pair one or more marker points of the instrument recognizable in the 2D x-ray images are extracted with an image processing algorithm. The image coordinates of these extracted points are assigned to one another. From these assignments a relative imaging geometry of the two recording systems can then be calculated so that the calibration of the two recording systems is completed. The extraction of a number of marker points of the instrument can be implemented for example with mapping catheters without additional effort.

The method is described below with specific reference to an endoluminal instrument. It can however of course be executed with other instruments, for example a biopsy needle.

For the calculation of the relative imaging geometry various pairs of images are required which are recorded at different spatial positions of the endoluminal instrument within the area under investigation and at identical imaging positions. The only point to note here is that the different positions of the instrument do not just lie in a straight line or in one plane but are spatially distributed in three dimensions. Especially advantageously the calibration described here can thus be undertaken for applications in heart chambers since in this case the catheter can be freely moved within the ventricle.

The calculation of the relative imaging geometry of the two recording systems from the point correspondences obtained can be undertaken using techniques such as those known from the area of computer vision. Such a method is for example disclosed in the publication by Z. Zhang, Determining the Epipolar Geometry and its Uncertainty: A Review, 1998, IJCV 27 (2), pages 161 to 198, which describes the determination of projection parameters of two central perspective imaging systems relative to one another from correspondence between points. With this method, which lies in the optical field, similar surface structures of an optical imaging of an object are automatically extracted from two images and placed in correspondence to one another. A corresponding feature in the second image is assigned to each feature from one of the images. For calculating the relative image geometry at least seven of these types of correspondence are required. However it is not possible to directly translate this method disclosed in the optical field to x-ray imaging technology, since by contrast with optical images, structures basically overlap in x-ray images and thus an automatic extraction of natural landmarks is prevented with x-ray images.

When more than two recording systems with different recording planes are used to calculate the relative imaging geometries between the different systems, a procedure can be used such as that described for example in B. Triggs, “Factorization Methods for Projective Structure and Motion”, CVPR'96, pages 845-851, or in the literature to which it refers.

With the present method by contrast the required correspondence between the points is obtained by moving the endoluminal instrument. Methods for automatic extraction of marker points of this type of instrument from the 2D x-ray image are known to the person skilled in the art. Thus for example the publication by I. H. de Boer et al., Methods for determination of electrode position in tomographic images, International Journal of Bioelectromagnetism, Number 2, Volume 2, September 2000, ISSN 1456-7865, shows a method for automatic extraction of a catheter tip from a 2D x-ray image.

If the catheter is now moved freely by the doctor, x-ray images can be simultaneously recorded in both recording planes of the device and the catheter tip extracted in each case. From each individual pair of recordings a point correspondence is thus obtained. Through successive acquisition of new recordings any number of point correspondences can be determined robustly within the shortest time without having to take special precautions for calibration. The method therefore makes do without calibration object or additional markers.

If the 2D x-ray images or projection images of the two recording planes are not taken simultaneously, that is at exactly the same point in time, but alternating to avoid the negative effect of stray radiation, then in an embodiment of the present method the position of the marker point in the 2D x-ray image of one of the two recording planes is suitably interpolated. This can be done by including one or more images recorded close to one another in time in the same imaging plane. The influence of patient movements on the calibration can be reduced by the interpolation.

The x-ray imaging device which is embodied for calibration in accordance with the present method features in a known way at least two recording systems for recording 2D x-rays of an area under examination in different recording planes as well as an evaluation and control unit for control of the imaging systems and evaluation of the image data obtained in the imaging systems. The outstanding feature of the x-ray unit is that the evaluation and control unit comprises a calibration module which from pairs of images made up of a first 2D x-ray image recorded with one of the recording systems and a second image recorded at the same time or immediately adjacent in time with the other recording system, extracts a marker point of an instrument recognizable in the 2D x-ray images assigns image coordinates of the extracted points of each pair of images to one another and from the assignments calculates and stores a relative imaging geometry of the two recording systems. With this x-ray imaging device the calibration of the two recording systems is thus undertaken via an image processing algorithm contained in the calibration module automatically after receipt of the first pair of images. The extraction of the marker points from the 2D x-ray images as well as the subsequent calculations can be undertaken in real time here.

The calibration sequence with this method is very simple and can be undertaken by the doctor during the intervention without additional effort. For the calibration in the preferred embodiment, software for calibration merely has to be started on the acquisition system, i.e. in the evaluation and control unit and the catheter subsequently moved freely for a few seconds in the area under investigation. The calibration can be undertaken here both before the intervention and also during the intervention. Before the intervention it is also possible to undertake the calibration without the patient by just moving the catheter within the recording volume covered by the recording system. A calibration during the intervention avoids the accuracy of the calibration depending on the reproducibility of the positioning of the C-arm. A particular advantage of the present method is also to be found in the fact that no additional aids have to be attached to the patient or to the catheter to enable online calibration to be undertaken. Instead the calibration is performed purely on the basis of the image data with known methods for extracting a marker point of the instrument especially for extracting the catheter tip.

### BRIEF DESCRIPTION OF THE DRAWINGS

The present x-ray device as well as the method for calibration are briefly explained again below with reference to an exemplary embodiment in conjunction with the drawings. The drawings show:

FIG. 1 an example of a biplane device which is embodied in accordance with the present invention;

FIG. 2 an illustration of the extraction and assignment of marker points of the endoluminal instrument in the 2D x-ray images; and

FIG. 3 an overview of the procedural steps for carrying out the present method.

### DETAILED DESCRIPTION OF INVENTION

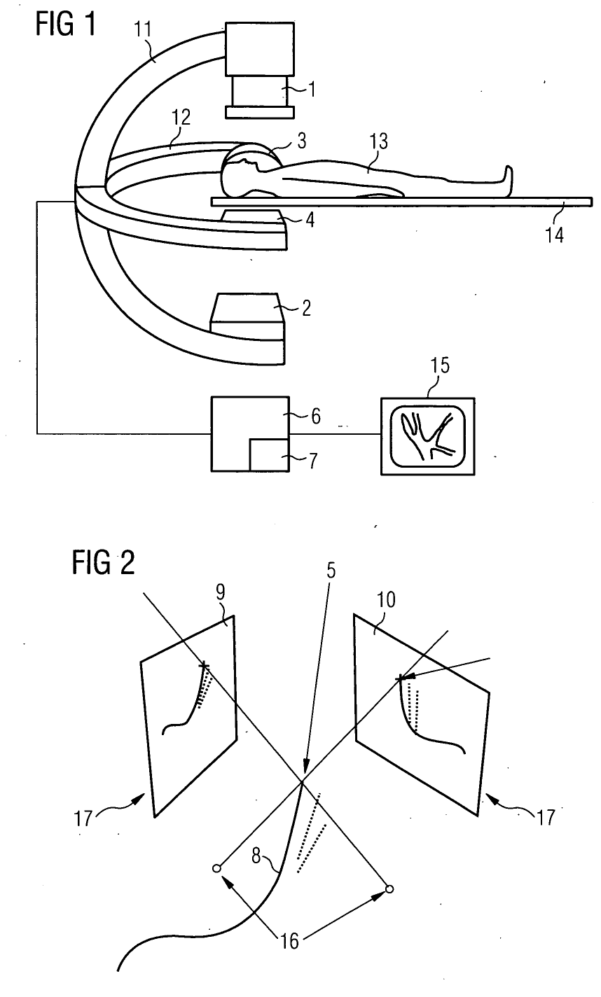

FIG. 1 shows an example of a biplane device with two C-arm systems 11, 12, which in the present example are arranged at an angle of 90° to one another. Each of the two C arm systems 11, 12 comprises a recording system with an x-ray tube 1, 3, as well as an x-ray detector 2, 4 arranged opposite the x-ray tube. With the two recording systems 1 to 4 2D x-ray images of the area under examination of a patient 13 who is supported on a patient bed can be obtained quasi-simultaneously in two different recording planes. The C-arms 11, 12 as well as the recording systems 1 to 4 are controlled via the control and evaluation unit 6 in which the evaluation of the image data for the presentation on a monitor 15 is also undertaken. The evaluation and control unit 6 also includes a calibration module 7 via which the calibration of the two recording systems 1 to 4 is undertaken in accordance with the method described below.

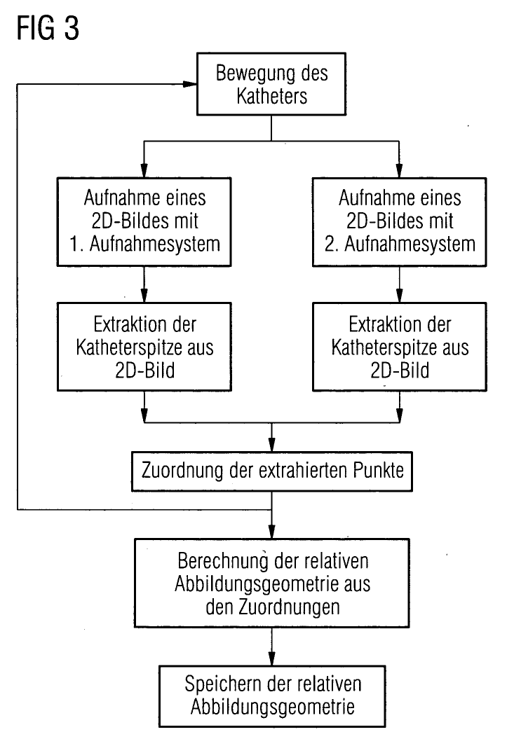

The basic execution sequence can be seen from FIG. 3. For the calibration the doctor in the present example moves a catheter to different spatial positions within the area under examination. At the different positions a 2D x-ray image is recorded practically simultaneously with both recording systems of the area under examination in which the catheter tip is recognizable. Subsequently an extraction of the catheter tip with an image processing algorithm is undertaken in both the recorded images. The points or coordinates obtained in the two images by this algorithm are assigned to each other. If enough assignments of different positions of the catheter are already available the relative imaging geometry of the two recording systems is calculated from the number of assignments and this relative imaging geography is stored. For unique calculation of the relative imaging geography at least seven assignments at different spatially distributed positions of the catheter are required. If there are not yet enough assignments available another movement of the catheter is made with the corresponding procedural sequence described.

FIG. 2 shows by way of an example and greatly schematicized the extraction and a assignment of the catheter tip from the two 2D x-ray images of an individual pair of images. In the figure at the projections centers 16 or as well as the detector planes 17 can be seen, which are produced by the arrangement of the x-ray tubes 1, 3 and detectors 2, 4 of the two recording systems and the relevant position of the two C-arms 11, 12. In the center of the image in the area under examination a catheter 8 is shown which is projected into the detector planes 17 by the recordings from the two different perspectives with the two recording systems. Corresponding 2D x-ray images 9, 10 in which the catheter 8 is visible are thus obtained in the detector planes 17. The projection of the catheter tip 5 on to these detector planes, i.e. the position of the catheter tip in the 2D x-ray images, is indicated in the figure by the crosses. This tip extracted by an image processing algorithm from the 2D x-ray images 9, 10 and the associated image coordinates are assigned to one another.

The same procedure is performed for a number of positions of the catheter within the area under examination, of which two more are indicated in FIG. 2 by the dashed lines. From the point correspondences obtained in this way the relative imaging geometry of the two recording systems can be calculated, especially the relative imaging scale as well as the angle of the recording planes to one another.

1.-5. (canceled)

6. A method of calibrating an x-ray imaging device having a first and a second image recording unit arranged for recording images relative to a first respectively a second image plane, the method comprising:
recording a first and a second two-dimensional image of an examination area of a patient by the first respectively second image recording units during an examination or treatment of the patient, the examination or treatment involving application of a medical instrument to the patient;
identifying a part of the medical instrument represented in both the first and second two-dimensional images by an image processing algorithm;
determining first coordinates of the part relative to the first two-dimensional image;
determining second coordinates of the part relative to the second two-dimensional image;
relating the first coordinates to the second coordinates; and
determining an imaging geometry of the first and second image recording units using the related first and second coordinates.

7. The method according to claim 6, wherein the first and second two-dimensional images are recorded substantially simultaneously.

8. The method according to claim 6, wherein recording the first and second two-dimensional images includes a delay in between the recordings.

9. The method according to claim 6, wherein the x-ray imaging device is a biplane x-ray imaging device.

10. The method according to claim 6, wherein the medical instrument is an endoluminal instrument.

11. The method according to claim 10, wherein the endoluminal instrument includes a catheter.

12. The method according to claim 6, wherein the recording of the first and second two-dimensional images is repeated after a change of position of the medical instrument relative to the examination area during the examination.

13. The method according to claim 6, wherein the part of the medical instrument is a tip of the medical instrument.

14. The method according to claim 6, wherein the part of the instrument is identified in real time.

15. The method according to claim 14, wherein also the determining of the first coordinates of the part relative to the first two-dimensional image, the determining of the second coordinates of the part relative to the second two-dimensional image, the relating of the first coordinates to the second coordinates, and the determining of the imaging geometry of the first and second imaging units are executed in real time.

16. The method according to claim 8, wherein determining the first or second coordinates include an interpolation.

17. An x-ray imaging device, comprising:
a first and a second image recording unit arranged for recording images relative to a first respectively a second image plane and adapted to record a first and a second two-dimensional image of an examination area of a patient by the first respectively second image recording units during an examination or treatment of the patient, the examination or treatment involving application of a medical instrument to the patient; and
a control unit for activating the image recording units and for processing image data acquired by the image recording units; and
a calibration unit adapted to:
identify a part of the medical instrument represented in both the first and second two-dimensional images by an image processing algorithm;
determine first coordinates of the part relative to the first two-dimensional image;
determine second coordinates of the part relative to the second two-dimensional image;
relate the first coordinates to the second coordinates; and
determine an imaging geometry of the first and second image recording units using the related first and second coordinates.
